(19) United States
(12) Patent Application Publication
(10) Pub. No.: US 2006/0264710 A1
(43) Pub. Date: Nov. 23, 2006

(54) Surgical method and apparatus using suction to hold tissue

(76) Inventor: Donald Spector, New York, NY (US)

Correspondence Address: Glen M. Diehl;NORTON & DIEHL LLC, Suite 110, 77 Brant Avenue, Clark, NJ, 07066, US

(21) Appl. No.: 11/175,638

(22) Filed: Jul. 6, 2005

Related U.S. Application Data
(60) Provisional application No. 60/676,320, filed on May 2, 2005.

(51) Int. Cl. A61B 1/32

(52) U.S. Cl. 600/201

(57) ABSTRACT

A new surgical device, system and methodology are disclosed. The surgical system includes a tube, a tip, a vacuum generator, a processing circuit and an interface. The processor determines how much vacuum should be applied through a tube and tip being used to achieve the desired holding power on a body part, such as an organ, without risking ripping of the body part. The calculation performed by the processor can be based on a number of parameters that can be entered through the interface. The parameters include the type of surgery, the body part being operated on, the strength of the body part, the viscosity of the body part, the particular tube being used including the diameter of the opening in the tube and the particular tip being used including the diameter of the opening in the tip. Another parameter is the age of the patient.

### CROSS-REFERENCE TO RELATED APPLICATIONS

This application claims the benefit of U.S. Provisional Application No. 60/676,320, filed May 2, 2005, which is incorporated herein by reference.

### BACKGROUND OF THE INVENTION

The present invention relates to surgical instruments, systems and methodologies.

Historically, surgical instruments have been made of solid materials that clamp onto, or are specifically designed for, the tissue to be moved. For purposes of the following disclosure, the term “tissue” is intended to include organs, bones, veins, arteries, entire body parts, components of body parts and the like. Although present surgical instruments are suitable for many applications, they can also present difficulties in many situations and have limitations. In particular, today's surgical instruments do not perform well where the surgery only allows small places in which to operate or where a great deal of delicacy is required.

Surgical instruments are designed to “grip” around an area to either cut or manipulate a structure. This means that out of necessity the area that goes around the structure must take up more room, in what often is a tight space, and thus requiring an incision greater than is necessary.

As an example, assume that one wanted to extend the area inside a spinal canal, surrounded by vertebrae, that has a disc that has moved inside the canal. Such surgeries are very serious and require a great deal of delicacy. It is typically difficult and dangerous to use conventional equipment to perform such a surgery, since the surgeon himself would have no way of “gripping” either the vertebrae or the disc and moving them from the inside out. The prior art for such procedures involves removing the disc and replacing it with a plug in fusion. While this procedure has had limited success in lower spinal areas, it is a dangerous and difficult procedure, particularly in higher cervical areas.

Another example would be where a growth would be removed from a delicate area. Typically, a surgeon would have to go in and remove a larger mass, which requires a relatively larger incision, increasing the odds of breaking other tissue barriers and increasing the risk of infection.

Accordingly, new and improved surgical tools, systems and methods are needed.

### SUMMARY OF THE INVENTION

The present invention provides a suction-based system for gripping and manipulating tissue, including body parts, during surgery, as well as removing excess tissue. In one aspect of the present invention, the system comprises a motor or other means of creating a vacuum. In accordance with one aspect of the present invention, the motor is controlled by an interface that provides a vacuum at a manipulating device. The manipulating device includes a tube and can include a tip. The interface means enables a user, such as a surgeon, to select the type of surgery, the type of tissue or body parts that are being operated on, the condition of the body part, the age of the patient as well as the particular instruments being used. The interface supplies the information to a circuit that uses the information to determine a corresponding vacuum value.

During surgery, the tube or, if used, the tip is placed against the body part being operated on and the motor is activated to generate a vacuum to hold the body part. The tube can be moved to manipulate the body part during the surgery, thereby allowing the surgeon the ability to manipulate the body part as necessary.

The tube and the tip can be any size needed. Thus, if the body part is small, the tube and the tip can be likewise small. In accordance with another embodiment of the present invention, the tip is shaped according to the body part being operated on.

The motor preferably generates a pressure that allows the body part to be held and manipulated without damaging the body part. In accordance with one aspect of the present invention, information about the surgery, the body part, the tube and the tip, the age of the patient and the condition of the body part are entered into the interface. The interface delivers this information to a circuit that uses the information to determine the appropriate vacuum. In accordance with a further embodiment of the present invention, the interface also permits the surgeon to manually increase or decrease the vacuum in accordance with the conditions encountered during the surgery.

The tube is connected to the motor. In accordance with one aspect of the present invention, the tube is flexible but retains its position when flexed. In one embodiment, fixing the position of the tube is performed by way of a stabilization device that may clamp onto other body structures, such as by suction, and which provides a locking mechanism for locking onto a portion of the tube to fix the extension and/or angle of the tube. The stabilization device may be a grid plate with an eyelet that allows manipulation of the eyelet which can be locked in place from the platform above the surgery. The tube is preferably made of titanium or steel, although other materials can be used. A tip is preferably adapted to the tube to attach to a body part. The tip is preferably made from a flexible material, such as rubber, latex, silicone or the like, and may have a shape that is adapted to both the type of tissue that is to be manipulated, and the type of manipulation that is to be performed. A variety of tips may be provided with the present surgical system, each tip being adapted for a particular surgery on a particular body part.

The vacuum means may provide multiple, independently controllable vacuum ports to which multiple manipulating means may individually be attached. The interface means may enable a user to individually control the pressure at each of the vacuum ports. Consequently, the user may simultaneously use multiple manipulating means to work with multiple tissue types. The interface means may provide identification coding, such as color coding, which corresponds to identification coding on the manipulating means to provide easy association of each manipulating means with its corresponding vacuum pressure value.

Each vacuum port may have a corresponding control switch for individually turning vacuum pressure on and off for that vacuum port. The control switch may be a physical switch located on the vacuum means or the interface means, or may be a virtual switch located on the interface means. Additionally, a master switch may be provided that simultaneously controls suction at all of the vacuum ports. The master switch may be a physical switch located on the vacuum means or the interface means, or may be a virtual switch located on the interface means.

An activation switch may be provided to control the application of suction at the tip of the manipulating means. The activation switch may be active-on, so that suction is always provided at the tip unless the activation switch is activated. Alternatively, the activation switch may be active-off, so that suction is provided at the tip only when the activation switch is activated. The activation switch may be disposed on the manipulating means so that the activation switch may be easily activated while the user is manipulating the manipulation means.

In one embodiment, the interface means may comprise a plurality of buttons or dials for selecting tissue type, manipulation type or vacuum pressure value. These buttons or dials may connect to a mechanical or electromechanical calculator that generates the vacuum pressure value in the form of an electrical or mechanical output for feeding into a corresponding input on the vacuum means. In another embodiment, the interface may comprise a processor, a display and input means. The processor controls the display to present a user interface comprising tissue types and manipulation types. The processor also obtains input from the input means. The user uses the input means to select tissue types, manipulation types or vacuum pressure values as presented on the display. The processor applies these user selections to a conversion algorithm to generate the vacuum pressure value, which is then electrically transmitted to the vacuum means. The input means may be a mouse, trackball, keyboard, touch-sensitive surface on the display, or a combination thereof.

A surgeon may utilize the interface means to enter information about the tissue type into the conversion means to generate the vacuum pressure value. Alternatively, the surgeon may use the interface means to enter the vacuum pressure value directly. The surgeon then positions the manipulating means so that the tip comes into contact with the desired tissue type. The vacuum means generates a vacuum at the tip to hold the tissue. The surgeon may optionally activate and deactivate the activation switch to grab and release the tissue.

One aspect of the present invention enables the user to grab and manipulate tissue by way of suction without damaging the tissue. Another aspect of the present invention allows removal of certain tissue by controlling the maximum suction applied according to tissue type. The level of suction force can be further controlled according to the type of manipulation that is to be performed to ensure that the minimal amount of suction necessary is applied to avoid tissue damage. Alternatively, high-strength tubing and appropriately designed tips allow an enormous amount of suction to be brought to bear at suitable tissue points, such as bone, to provide an anchoring system.

Another aspect of the present invention is to provide a single point of connection to tissue or body parts through suction, thereby allowing the operation to be performed in smaller places. A further aspect of the present invention is to allow the insertion of a probe with a diameter smaller than a growth that is to be removed and to provide high enough suction that the mass to be removed undergoes a phase change. In an extreme case, a golf ball could be suctioned through a small needle. For example, during certain types of ultrasonic surgery, where kidney stones are broken up, problems have arisen since the stones may break up but are unremoved from the system often causing infection or damaging remaining structures. These broken up stone may be removed in accordance with one aspect of the present invention.

### DESCRIPTION OF THE DRAWINGS

FIG. 1 illustrates a block diagram of a surgical system in accordance with an aspect of the present invention.

FIG. 2 illustrates the system of FIG. 1 being connected to a vertebra with a plate during a surgical procedure.

FIGS. 3 to 5 illustrate a surgical system in accordance with various aspects of the present invention.

### DESCRIPTION OF THE PREFERRED EMBODIMENT

The present invention provides a new line of surgical instruments, systems and methodology. In accordance with one aspect of the present invention, the surgical system creates a vacuum to provide a precise amount of suction to enable surgeons to hold and move bones, organs or other body parts in humans or animals by the application of the vacuum to the bones, organs or other body parts that results in measured adhesion forces. Also, other types of excess tissue or materials may be manipulated. For example, in certain types of ultrasonic surgery, such as where kidney stones are broken up, problems have arisen since the stones may break up but are not removed from the system, often causing infection or damage to remaining structures. The present invention allows for easy and precise manipulation and removal of such stones and their fragments.

The surgical instruments are made of very fine tubing capable of supporting high suction values. Materials such as titanium, steel or other similar materials are preferred for the tubing. This tubing can be of microscopic proportions, though in common practice might be longer or wider. The calculations of the adhesive force created by the vacuum will be measured against the viscosity of the tissue being held and moved so as not to rupture the organ or area being gripped by the suction, except in those cases where this is specifically an objective of the surgery.

In accordance with an embodiment of the present invention, the suction tubing is provided with a soft latex, rubber or silicone tip that is attached to steel, titanium or other tube capable of supporting high levels of suction.

One end the tubing is connected to a box that may be controlled by a circuit that may, for example, include a computer, and that calculates the viscosity and maximum pressure per surface area for the tissue that is to be “gripped” by the tip connected to the tubing. Therefore, the lower the viscosity and the “breaking point” of the tissue, and the more pressure that is needed to move said tissue, the smaller the diameter of the tubing. This is reflected by the size of the aperture of the tubing, and the pressure required which is sufficient but cannot exceed the pounds per square inch that would rupture or cause damage to the tissue being moved, except in those parts of the surgery in which specific parts of tissue may wish to be removed. In summary, the amount of vacuum can be controlled in accordance with the surgery being performed, in accordance with information concerning the tube and the tip, in accordance with the body part being operated on and in accordance with the actual condition of the body part being operated on.

The control mechanism may be connected to a microprocessor capable of storing the viscosities of the tissue being gripped onto and recommending the maximum tubing necessary to develop the pressure to grip such an organ, without rupture. There are obviously certain situations in which this would not be possible to accomplish.

The present discussion of the equipment functioning will be in the area of neuroanatomy, though the claims for use of this process will extend beyond the scope of the present embodiment. Consider the surgery previously mentioned where a surgeon needs to extend the area inside a spinal canal surrounded by vertebrae that has a disc that has moved inside the canal. It would typically be difficult and dangerous to use conventional equipment to perform such a surgery, since the surgeon himself would have no way of “gripping” either the vertebrae or the disc and moving them from the inside out. The prior art for such procedures involves removing the disc and replacing it with a plug in fusion. While this has had limited success in lower spinal areas, it a dangerous and difficult procedure, particularly in higher cervical areas. In accordance with one aspect of the present invention, a plastic clear plate would be manually secured to the outside of the cervical vertebrae. This, too, can be secured by the suction provided by the microprocessor that is controlling the instrumentation. This plastic plate would have a series of holes in it, such that the new surgical tool (i.e., the tubing) could be inserted to connect to the vertebrae or disc at a variety of locations and with a variety of diameters. The holes in the plastic cover may have eyelets that can be rotated but that have stops that limit and lock in the distance to which the tubing can be inserted through the plastic plate to come into contact with the vertebrae.

The tubing, with the flexible tip, may be inserted to grip the vertebrae. The suction would be great enough to enable the surgeon, through the precise eyelets, to pull the vertebrae in any locked in axis towards the plastic plate and away from the spinal cord. A separate tube may suck onto an inside disc and may contain the precise pressure capable of moving the disc, without rupturing or sucking in the disc. This would be computer determined based on the strength and viscosity of the disc, and the diameter and amount of suction needed in order to move the disc.

Such a surgical tool would enable a neurosurgeon to grip onto virtually any part of a bone structure, or organ, with as small a tubing as needed and pull that area out, without the need to enter the canal that is not affected by the surgery.

Suction has long been used in surgery, mostly to take away excess blood and tissue. In this case the suction devices envisioned are of a different generation of power, providing several thousand times the normal suction, but allowing a surgeon to know through computer technology exactly how much force can be applied to a structure without destroying the integrity of the structure. However, it should also be appreciated that this equipment would be able to accomplish and amplify the demands of normal suction by providing more power than has ever been used in surgical suction before, thereby enabling the removal of larger structures through the suction of small instrumentation.

The total suction force needed may also relate to the amount of force that is needed to physically manipulate the structures that are held in place by muscles and or other connective tissue, ligaments etc. In most cases, the suction that would be adapted so that a minimal number of these supporting areas would have to be cut. The computer would define the risk reward based on the size of the tubing that was possible to fit into the area, and the viscosity and tensile strength of the tubing would be correlated with the force required of the object to be moved.

In the present example the surgery would then consist of opening the neck and the insertion of a plastic plate that would be secured over several vertebrae. The surgeon would then simply insert the new suction surgical tube, and latch onto any structure needed, with the proper angle being defined by the articulated eyelet. The structure may be moved externally, causing the freeing up on the intrusion from the outside, those structures could then be fused into place, while the suction was on. By turning off the suction the instruments may immediately be released and the surgery completed, using this assisted computer control system in a matter of minutes.

This system is applicable not only in medical areas, but may also be used for delicate wiring problems, or other areas where a small (possibly microscopic) probe is automatically capable of gripping a structure on one side and moving that structure without having to envelop the item as a conventional clamp or clip might have to do. By hitting one spot on one axis, the suction tool provides a way of entering delicate environments and obtaining a strong temporary grip that is capable of precise manipulation in a multiple axis environment, while actually controlling the field of motion from a single axis.

The linkage of the tubing which can go from microscopic to macroscopic with flexible tubing capable of gripping without breaking the tissue, or disrupting the viscosity of the tissue.

Thus, the surgical system of the present invention provides the ability to move the object or tissue with precision on a multiple axis basis from a strong but temporary single point that is easily accessed.

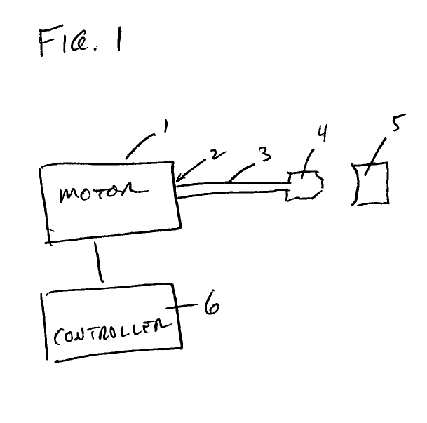

A block diagram of the present system is illustrated in FIG. 1. A motor 1 generates a vacuum at its output 2. A tube 3 is connected to the output 2 and a tip 4 is connected to the end of the tube 3. The motor 1 generates a vacuum that is present at the output of the tip 4. The tip 4 is connected to a body part 5 that is being operated on. In operation, the vacuum generated by the motor 1 is applied to the body part 5 through the tube 3 and tip 4 to hold the body part 5. The tube 3 is flexible and can be moved to a new position to manipulate and move the body part 5 as needed.

A controller 6 is connected to the motor 5. The controller 6 controls the operation of the motor 5, including the amount of vacuum created by the motor 5. The controller 6 preferably includes an interface and a control circuit. The interface includes standard personal computer interface devices such as a keyboard, display and mouse. The display can be responsive to touch. The circuit can be a special purpose circuit or can include a microprocessor.

In operation, information concerning the surgery being performed is entered into the interface of the controller 6. The information includes the surgery being performed, the body part being held and manipulated, the condition of the body part, the age of the patient and information concerning the tube and the tip. The circuit in the controller 6 uses this information to determine the vacuum needed to hold and manipulate the body part 5 without damaging the body part 5. The circuit preferably has a memory that holds a look up table that generates instructions to the motor 1 based on the information entered. The controller 6 also preferably has manual controls that can be used to increase or decrease the vacuum generated by the motor 1.

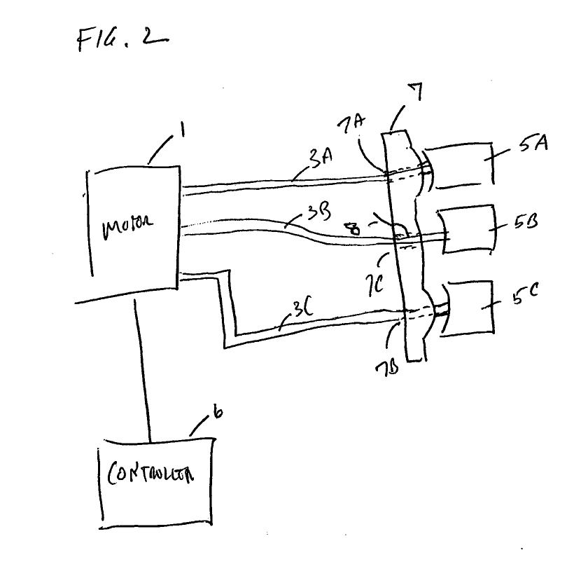

A system in accordance with a further aspect of the present invention is illustrated in FIG. 2. In this case, the motor 1 supplies a vacuum to multiple tubes 3a, 3b and 3c. The vacuum supplied at each tube 3a, 3b and 3c are preferably independently controlled according to the particular needs of the surgery being performed.

Tubes 3a and 3c are connected to ports 7a and 7c, respectively, in a device 7. The device 7 is preferably plastic and is shaped in accordance with the surgery being performed. For example, if a vertebra 5b is being operated on, the device 7 is designed to interface with the vertebrae 5a, 5b and 5c.

Referring to FIG. 2, the motor 1 supplies a vacuum through tubes 3a and 3c, through the ports 7a and 7c such that the device 7 is secured to the vertebrae 5a and 5c. The tube 3b is placed through the port 5b and in contact with the vertebra 7b. The tube 3b is flexible and can be moved relative to the device 7 with a vacuum applied to the vertebra 7b to manipulate the vertebra 7b. A locking mechanism 8 can be activated, in this case by depressing a lever, to lock the tube 3b in a desired position.

The controller 6 can be used to individually control the vacuum applied to each of the tubes 3a, 3b and 3c.

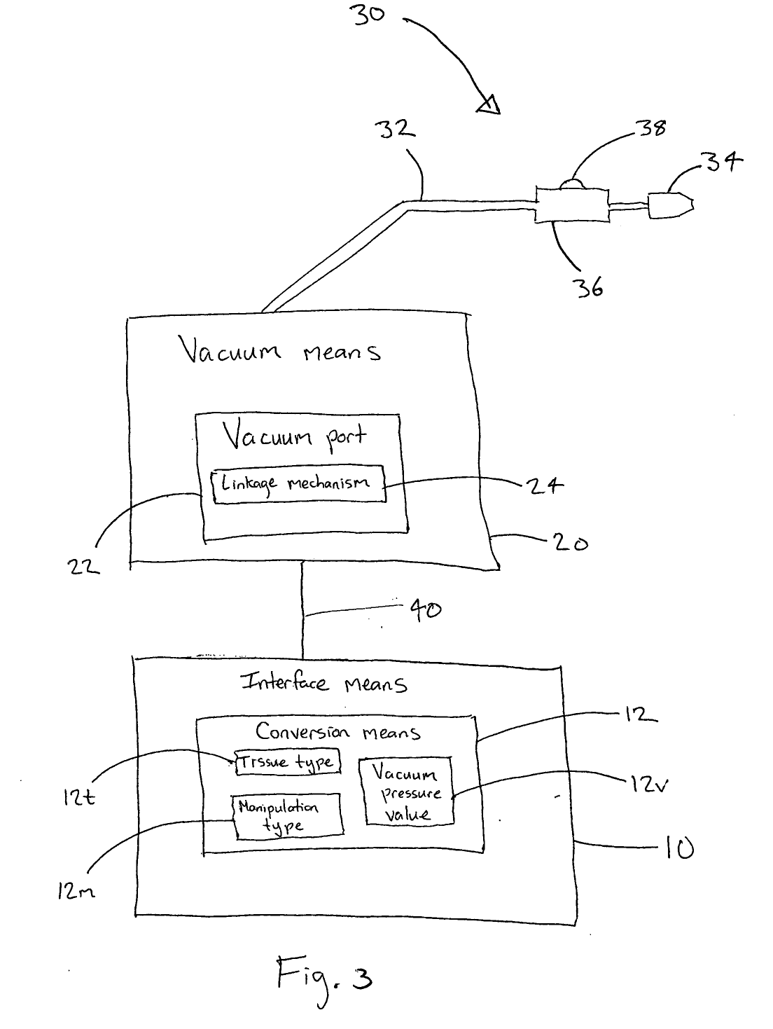

A system in accordance with another aspect of the present invention is illustrated in FIG. 3. The system includes an interface 10, a vacuum 20 and a manipulator 30. The interface 10 provides a man-machine-interface (MMI) for the system. The interface 10 may include a display, a keyboard, a mouse and a conversion device 12. The interface 12 enables a user to select a tissue type 12t, and optionally a manipulation type 12m, to generate a vacuum pressure value 12v. The interface 10 enables the user to enter the vacuum pressure value 12v; alternatively, the interface 12 enables the user to enter the tissue type 12t and the optional manipulation type 12m, and generates the vacuum pressure value 12v according to the tissue type 12t and manipulation type 12m. Other parameters that can be entered through the interface 10 includes the type of surgery being performed, the body part or tissue being operated on, the age of the patient, and the equipment being used. Design of the interface 10 should be well within the abilities of one with reasonable skill in the art.

The conversion device may be a look up table stored in memory in the interface. The look up table stores values associated with each of the particular parameters. The values determine the appropriate vacuum to be created by the vacuum. Of primary importance in determining the proper vacuum is the surgery being performed and the body part or tissue that must be held, manipulated or removed.

The vacuum value 12v determined by the conversion device is provided from the interface 10 to the vacuum 20 via a communication port 40.

The vacuum 20 inputs the vacuum pressure value 12v from the communications port 40, and generates vacuum pressure at a value that is effectively equal to the pressure represented by the vacuum pressure value 12v. The vacuum pressure is provided at a vacuum port 22.

The manipulator 30 may detachably connect to the vacuum port 22, and includes a tube 32 and a tip 34. One end of the tube 32 may detachably connect to the vacuum port 22. The tip 34 is attached to the other end of the tube 32. Consequently, the tube 32 provides to the tip 34 the vacuum pressure present at the vacuum pressure port 22. The manipulator 30 may be provided a handle 36 to provide a convenient grasping point for the user. The manipulator 30 may also be provided an activation switch 38 to toggle the presence of vacuum suction at the tip 34. The activation switch 38 may be disposed on the handle 36 for easy access, and may be active-on or active-off. The tip 34 may be changed according to the tissue type 12t and manipulation type 12m; that is, the tip 34 may be designed to interface with a specific type and shape of tissue. The tip 34 may be made of a flexible material, such as latex, rubber or silicone.

Many methods may be employed to provide the interface 10. Towards the simplest extreme, the conversion device 12 may be a written table that a user references to obtain the vacuum pressure value 12v. For example, rows corresponding to the tissue type 12t, and columns corresponding to the manipulation type 12m, could provide a grid of corresponding vacuum pressure values 12v. The user may select the appropriate vacuum pressure value 12v and manually enter it into the interface 10, by way of buttons, knobs, switches or the like; such entering would create a corresponding state change on the communications port 40 to communicate the value 12v to the vacuum 20. Alternatively, the conversion device 12 could actively generate the vacuum pressure value 12v from the tissue type 12t and manipulation type 12m entered by the user. Buttons, knobs switches or the like may be provided to enable the user to enter the tissue type 12t and manipulation type 12m. In this case, the conversion device 12 could be a computer, mechanical, electromechanical or digital, that takes the user-input tissue type 12t and manipulation type 12m and generates the vacuum pressure value 12v. The methods for performing such conversions are well known. At the more complex extreme, the interface 10 may be a computer system that provides an interactive user interface enabling the user to electronically control all aspects of the system, including the selection of tissue type 12t and manipulation type 12m for automatically generating the vacuum pressure value 12v, and the manual entering of the vacuum pressure value 12v based upon experience or intuition.

The tissue type 12t is a value that corresponds to the type of tissue that the tip 34 will contact, and may include, for example, liver, kidney, lung, heart, bone, etc., and sub-sets thereof Manipulation type 12m is a value that corresponds to the type of action the user expects to perform on the tissue with the probe 34, and may include, for example, fine movement, gross movement, stabilization, anchoring, extraction, etc. Generally, tissue type 12t sets the maximum amount of suction that should be provided at the tip 34; that is, a minimum value for the vacuum pressure value 12v. The vacuum pressure value 12v may then be adjusted up according to the manipulation type 12m to provide the least amount of suction required at the tip 34 to get the desired type of manipulation done. However, in the case of extraction, in which damage to the target tissue is irrelevant, the vacuum pressure may be adjusted beyond the otherwise maximum safe suction value.

The vacuum 20 may be any vacuum source that is capable of providing selectable, consistent and closely regulated vacuum pressures (differential or absolute) according to the communications port 40. Such vacuum systems include motors and are known in the art, and interfacing these vacuum systems with the MMI 10 via the communications port 40 is also well known. The vacuum port 22 may provide a linkage mechanism 24 that enables a well-sealed connection to the manipulator 30. The linkage mechanism 24 may provide for easy attachment and detachment of the manipulator 30. Such linkage mechanisms 24 are well known.

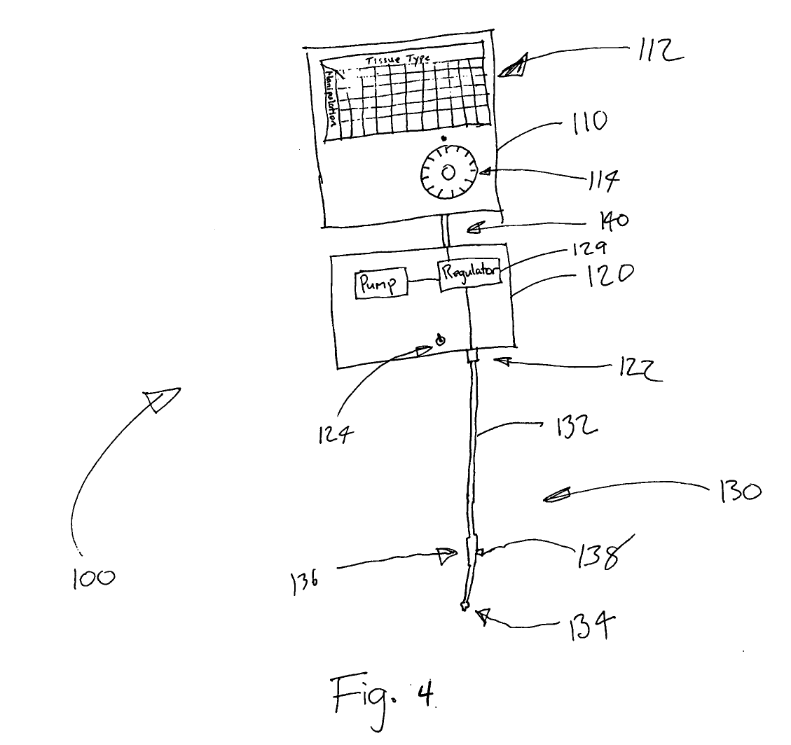

Referring to FIG. 4, a system 100 according to another aspect of the present invention is shown. The system 100 includes an MMI 110, and a regulated vacuum system 120 controlled by a mechanical linkage 140. The vacuum system 120 generates suction at a vacuum port 122 at a value that is controlled by the mechanical linkage 140, and may do so using a vacuum regulator 129. As the mechanical state of the linkage 140 changes, say by extension or rotation, so too in a corresponding manner does the value of the vacuum pressure at the vacuum port 122. The mechanical linkage 140 thus conveys a vacuum pressure value from the MMI 110 to the vacuum system 120. The mechanical linkage, in turn, is controlled by a dial 114; the dial 114 is calibrated so that each calibration mark on the dial 114 causes a corresponding vacuum pressure to be developed at the vacuum port 122. The MMI 110 includes a conversion chart 112. The chart 112 includes columns for tissue type, and rows for manipulation type. Each intersection of column and row contains a calibration value on the dial 114 that corresponds to appropriate suction values for the tissue and manipulation types. The user then uses the dial 114 to enter this calibration value, which in turn causes the appropriate suction to be developed at the vacuum port 122. The vacuum system 120 may include a switch 124 for turning vacuum pressure on and off at the port 122. Although depicted as separate units, the MMI 110 and the vacuum system 120 may be disposed in a single casing.

The vacuum port 122 permits detachable connection of a manipulator 130. The manipulator 130 includes a tube 132, one end of which detachably connects to the port 122 in any manner familiar to those in the art. The tube may be made of titanium, steel, rubber or any other suitable material that can withstand the vacuum pressure without collapsing. The other end of the manipulator 130 ends in a tip 134, which is placed into contact with the tissue. The tip 134 may be made of a resilient material, such as rubber, silicone or the like. The manipulator 130 may also be provided a handle 136, and an activation switch 138 to toggle the presence of vacuum pressure at the tip 134. The activation switch 138 may be active-on, so that suction is always provided at the tip 134 unless the activation switch 138 is activated, or the activation switch 138 may be active-off, so that suction is provided at the tip 134 only when the activation switch 138 is activated. The tip 134 may be conformal to the type of tissue and manipulation, so that different types of tips 134, and different types of tubes 132 may be used to form the manipulator 130.

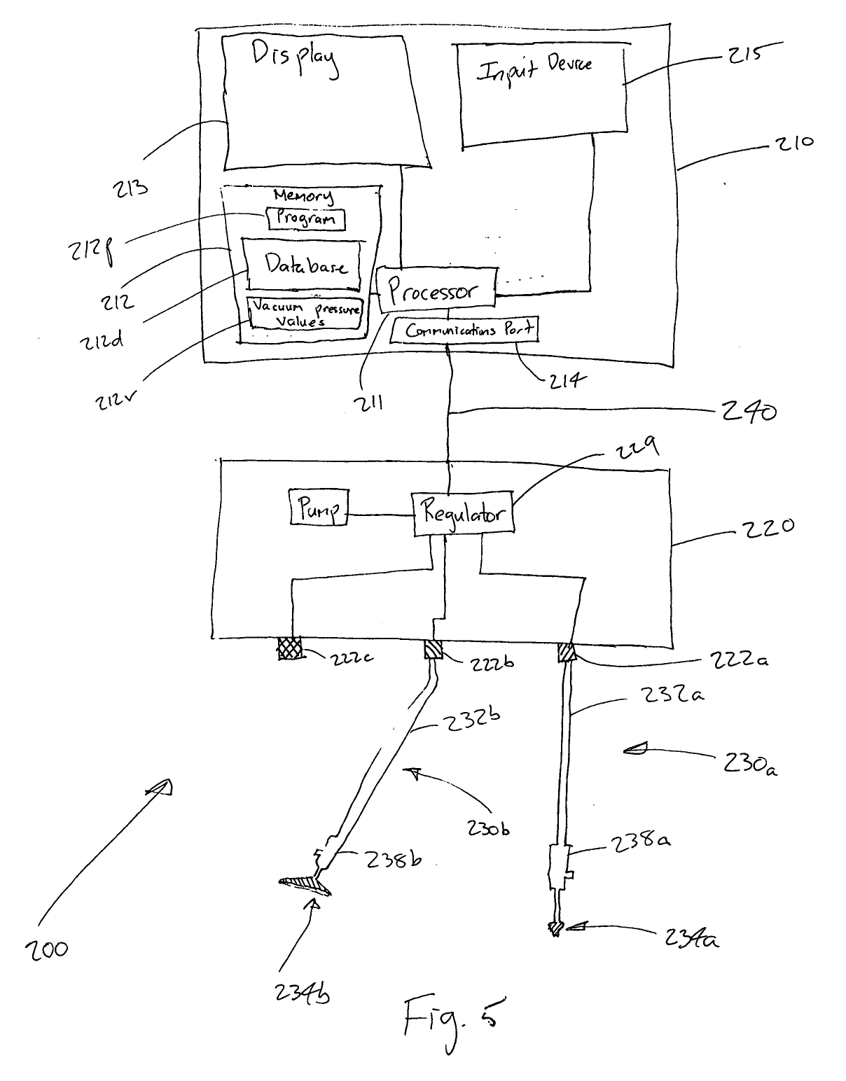

Referring to FIG. 5, a block diagram of a further aspect of the present invention is shown. A system 200 includes an MMI 210 that controls a vacuum system 220 via an electronic interface 240. The vacuum system 220 has a plurality of vacuum ports 222, each of which may independently provide suction at a unique vacuum pressure value. The vacuum pressure at each port 222 is controlled by a regulator 229, which in turn is controlled via the electronic interface 240. Each port 222 is provided a unique color for ready identification.

The MMI 210 is a computing system that includes a processor 211, memory 212, a display 213, a communications port 214 and one or more input devices 215. The display 213 may be a touch-sensitive screen, and hence may also serve as an input device. In addition to the display 213, the input device 215 may include a mouse, a keyboard, a trackball, or any other suitable input device. The communications port 214 is controlled by the processor 211, and is used to send instructions to the vacuum system 220 through the interface 240. These instructions tell the regulator 229 to set the individual pressures at each of the vacuum ports 222a-222c, or to selectively turn the ports 222a-222c on and off. The interface may be proprietary or common, such as RS-232, USB, parallel, etc.

The memory 212 includes a program 212p that is executable by the processor 211, and which controls the operations of the MMI 210; a database 212d that is used to convert user inputs into appropriate vacuum values, and vacuum pressure values 212v that individually hold the vacuum values desired at each of the vacuum ports 222. The vacuum values may be absolute or differential, though differential may be preferred. For example, if there are three vacuum ports 222a-222c, then the memory 212 would hold three corresponding vacuum pressure values 212v. The database 212d is a relational database that associates a vacuum value with tissue types, manipulations types, the type of tube 232 used in the manipulator 230, and the type of tip 234 used. Utilizing input provided by the user via the input device 215 and/or display 213, such as tissue type and manipulation type, the processor 211 utilizes the database 212d to generate a corresponding vacuum value. The user need not provide all relevant information to enable the processor 211 to look up a vacuum value in the database 212d. For example, the user need only provide the tissue type, in which case the database 212d would return the minimum safe vacuum value for such type of tissue (i.e., the maximum safe suction). By providing the type of manipulation being performed, this value could be nudged upwards or downwards, thereby reducing the suction present on the tissue at the tip 234 and thus the risk of inadvertent damage, or increasing the suction to provide for extraction at the safest pressure value. Each additional piece of information provided by the user simply assists the processor 211 to fine-tune the vacuum value. Alternatively, the user may directly enter a vacuum value, and no conversion is performed. This vacuum value is then used to set one of the vacuum pressure values 212v for a desired port 222. The processor 211 utilizes the communications port 214 to pass the vacuum pressure values 212v to the vacuum system 220 as required.

The display 213, controlled by the processor 211, provides a visual interface that enables the user to view and change the vacuum pressure values 212v, either directly or by conversion of tissue type, manipulation type, etc. The display 213 may present color coded information that corresponds to the color coding used on the vacuum ports 222. For example, if vacuum port 222a is coded red, then tissue type, manipulation type and the corresponding vacuum pressure value 212v for port 222a would all be presented in red to facilitate ready identification of port 222a with any data input and viewed by the user. Additionally, the display 213 may present color coded virtual switches to individually turn the vacuum ports 222 on and off, and a master switch to turn all of the ports 222 off simultaneously. Standard user interface features may be presented on the display to facilitate in the presentation and entry of information, such as menus, tabbed pages, drop-down boxes and entry fields.

Each vacuum port 222 may be provided a corresponding manipulator 230. The tips 234, tubes 232 and/or handles 238 may be color coded to match the color of the vacuum port 222 to which the respective manipulator 230 is attached. In this manner, ready identification of manipulator 230 to port 222 to corresponding visual information on the display 213 is ensured. Each manipulator 230 may be provided a corresponding activation switch 238, which may be active-on or active-off as required, to toggle the presence of suction at the tip 234.

Although the invention herein has been described with reference to particular embodiments, it is to be understood that these embodiments are merely illustrative of the principles and applications of the present invention. It will be apparent to those skilled in the art that various modifications and variations can be made in the apparatus and methods of the present invention without departing from the spirit and scope of the invention. Thus, it is intended that the present invention include modifications and variations that are within the scope of the appended claims and their equivalents.

1. A surgical system for performing an operation on a body part, comprising:
vacuum means for creating a vacuum at an output of the vacuum means;
a tube having a hollow section between a first end and a second end, the first end of the tube being connected to the output of the vacuum means so that the vacuum can be provided at the second end;
a tip attached to the second end of the tube so that the vacuum can be provided at an output of the tip;
an interface that receives information concerning the operation; and
a circuit connected to the vacuum means and to the interface, the circuit receiving information concerning the operation from the interface, determining an appropriate vacuum and controlling the vacuum means to provide the appropriate vacuum.

2. The surgical system as claimed in claim 1, wherein the interface includes a display and a keyboard.

3. The surgical system as claimed in claim 1, wherein the tube is flexible.

4. The surgical system as claimed in claim 1, wherein the tube is titanium.

5. The surgical system as claimed in claim 1, wherein the tube is steel.

6. The surgical system as claimed in claim 1, wherein the tip is rubber.

7. The surgical system as claimed in claim 3, wherein the tip is rubber.

8. The surgical system as claimed in claim 1, wherein the tip is shaped according to the body part being operated on and the tip is removable from the tube.

9. The surgical system as claimed in claim 1, wherein the circuit includes a microprocessor.

10. The surgical system as claimed in claim 1, wherein the circuit determines the appropriate value for the vacuum in part based on the operation.

11. The surgical system as claimed in claim 10, wherein the circuit can receive information concerning the tube and the tip and can determine the appropriate value for the vacuum in part based on the information concerning the tube and the tip.

12. The surgical system as claimed in claim 10, wherein the circuit can receive information concerning the body part and can determine the appropriate value for the vacuum in part based on the information concerning the body part.

13. The surgical system as claimed in claim 1, wherein the interface circuit can receive instructions to increase and to decrease the vacuum and the circuit controls the vacuum means in accordance with the instructions.

14. A method of performing surgery on a body part in a body, comprising:
positioning an end of a tube against a body part;
creating a vacuum in the tube to hold the body part.

15. The method as claimed in claim 14, further comprising moving the tube to move the body part.

16. The method as claimed in claim 15, further comprising:
entering information concerning the surgery into a circuit;
the circuit using the information to determine the vacuum needed to hold the body part without damaging the body part.

17. The method as claimed in claim 1, wherein a tip is connected between the tube and the body part.

18. The method as claimed in claim 17, further comprising selecting the tip in accordance with the body part being operated on.

19. The method as claimed in claim 14, further comprising increasing or decreasing the vacuum as needed.

20. A surgical system for performing an operation on a body part, comprising:
a motor having a vacuum at an output of the motor;
a tube having a hollow section between a first end and a second end, the first end of the tube being connected to the output of the motor so that the vacuum can be provided at the second end; and
a tip having an opening adapted to fit to the body part and being attached to the second end of the tube so that the vacuum can be provided at the opening of the tip.

21. The surgical system as claimed in claim 20, further comprising:
an interface on which information concerning the operation can be entered; and
a circuit connected to the motor and to the interface, the circuit receiving the information concerning the operation from the interface, determining an appropriate vacuum and controlling the motor to provide the appropriate vacuum.

22. The surgical system as claimed in claim 21, wherein the information includes the operation being performed.

23. The surgical system as claimed in claim 21, wherein the information includes the body part.

24. The surgical system as claimed in claim 23, wherein the information includes the condition of the body part.

25. The surgical system as claimed in claim 21, wherein the information includes the age of a patient being operated on.

26. The surgical system as claimed in claim 21, wherein the information describes the tube and the tip.
